(19) United States
(12) Patent Application Publication
(10) Pub. No.: US 2008/0255538 A1
(43) Pub. Date: Oct. 16, 2008

(54) HEATING CIRCUIT AND METHOD FOR INTRAVENOUS FLUID DELIVERY

(75) Inventor: Kent D. Ellis, Seattle, WA (US)

Correspondence Address: SEED INTELLECTUAL PROPERTY LAW GROUP PLLC, 701 FIFTH AVE, SUITE 5400, SEATTLE, WA, 98104, US

(73) Assignee: LMA Medical Innovations Limited, Victoria (SC)

(21) Appl. No.: 12/047,251

(22) Filed: Mar. 12, 2008

Related U.S. Application Data
(60) Provisional application No. 60/894,436, filed on Mar. 12, 2007.

(51) Int. Cl. A61M 5/44; H05B 1/00

(52) U.S. Cl. 604/507; 604/114; 607/105; 219/494; 392/441

(57) ABSTRACT

An apparatus for warming fluids includes a heating element having a resistance value that limits an amount of heat dissipated from the heating element to a threshold temperature value in response to a specific amount of electrical energy being applied to the heating element. A fluid carrying member may have the heating element coupled thereto to transfer heat from the heating element to a fluid within the fluid carrying member. A power supply may control a temperature of the fluid so as not to exceed the threshold temperature value by applying the specific amount of electrical energy to the heating element.

### BACKGROUND OF THE INVENTION

1. Field of the Invention

This description generally relates to the field of heating intravenous fluid, and more particularly to a method and apparatus of heating an intravenous fluid to the proper temperature without going above a safe temperature.

2. Description of the Related Art

Intravenous fluid delivery, such as drug delivery, is ubiquitous in healthcare practice throughout the world. Intravenous fluid delivery is an important element in the ATLS (Advanced Trauma Life Support System) protocol for management of trauma victims. The ATLS protocol is designed to maximize treatment in the first “golden hour” which immediately follows a trauma, and thus ensure an optimal long-term outcome. Treatment during the “golden hour,” which typically includes intravenous fluid delivery, may commonly occur on battlefields, in hospitals and in ambulances.

One common need for heated IV fluids is during treatment of hypothermia. A common cause of hypothermia is surgery, which typically leads to mild core hypothermia. There exist a number of studies that have demonstrated that mild peri-operative hypothermia results in a number of adverse outcomes. Mild hypothermia may cause myocardial ischemia and arrhythmias, increases intra-operative blood loss and the requirement for blood transfusion. Mild hypothermia also predisposes to surgical wound infection and poor wound healing. There are three basic strategies for the prevention and treatment of mild peri-operative hypothermia: minimizing heat loss, cutaneous warming and internal warming. Studies have shown a reduction of body core temp of 0.9+/−0.2° C. when IV fluid was administered at room temperature during abdominal surgery. Further studies have shown a reduction in core temp of 0.25° C. for every liter of IV solution administered at room temp (21° C.). To minimize heat loss during general anesthesia, IV fluids are warmed to body temperature prior to infusion.

A danger which must be avoided is heating the fluid above too high of a temperature. If the fluid temperature is above 42° C. and is input directly to the patient, there is a high likelihood that substantial damage and harm may be done. Fluid temperatures in the range of 40° C. to 42° C., while not so dangerous, are likely to be unhealthy. A preferred temperature range for the input of fluid is in the range of 37° C.-39° C., and an acceptable range is 30° C.-37° C. Some prior art techniques, such as heating in a microwave oven, or warming with a heating pad, have potential of locally overheating some portions of the fluid. Such systems would normally require complex feedback sensors to monitor the fluid temperature or the temperature of the heater to be sure that the intravenous fluid does not exceed a desired temperature, yet such feedback sensors are not easily provided on such methods of heating.

Current fluid warming devices typically include a reservoir of heated fluid that is delivered to a patient or a pair of heating plates in close proximity to the fluid bag (e.g., IV bag). Examples of some fluid warming devices are: the HOTLINE®, the RANGER®, the Bair Hugger® and the Fluido®.

The Hotline is a coaxial system where the administered fluid flows through an inner lumen. The fluid is warmed using the counter flow of fluid traveling from a warming device along an outer lumen. The Hotline device utilizes a reservoir of heated water that is pumped and circulated around an IV tubing, which carries fluid to a patient.

The warming device disclosed in U.S. Pat. No. 5,875,282 teaches an IV bag located between two hot plates that produce specific levels of heating. IV fluid lines flow through the IV bag that contains an elongated path for the flow of fluids. The IV bag carrying the fluids is in contact with the two hot plates and conductive heating takes place as the fluids flow through the elongated circuitous path of the IV bag. Fluid temperature is measured by a sensor that is in contact with the IV bag and positioned at an outlet of the bag. The design as taught in U.S. Pat. No. 5,875,282 is also found in the RANGER® product.

The Bair Hugger has a coil placed within a hose of a forced air warming device.

The Fluido incorporates a flat rigid plastic cassette that is placed in a heating pod. The Fluido uses infrared lamps to heat the fluid. The cassette has a priming volume of 90 ml and incorporates flow sensors. The heating pod has software that adjusts the heat energy given to the fluid with varying flow rates so as to deliver fluid at a predetermined temperature.

Current products have a distance from a site of warming to the percutaneous point of insertion of the IV line into the patient. Such distance reduces the effectiveness of fluid warming since the temperature of the fluid in the tube is influenced by the outside temperature while flowing from the site of warming to the percutaneous point of insertion of the IV line. For example, fluids traveling in an IV tube may be under the influence of the temperature in the room (e.g., 20° C. or 70° F.) while flowing from the site of warming to the percutaneous point of insertion, which adversely affects the fluid temperature in the tube. IV administration may be necessary in harsh environmental conditions (e.g., outside area temperature below 0° C. or 32° F.), thereby increasing the adverse affect of the distance from the site of warming to the percutaneous point of insertion of the IV line into the patient.

Some existing warming devices require sensors positioned downstream the site of warming (e.g., proximate the percutaneous point of insertion) to determine the actual fluid temperature entering the patient's body. A controller may be typically used in conjunction with the sensors to adjust the temperature of the heating elements at the warming site so as to more closely achieve a desired temperature of fluid that enters into the patient. The controller may also assure that a maximum or minimum temperature is not reached. Furthermore, additional equipment such as a controller and sensors are costly to manufacture and complicate the design of the warming device.

It is therefore desirable to have a fluid warming device that addresses or alleviates at least some of the above stated problems.

### BRIEF SUMMARY OF THE INVENTION

According to one embodiment, a device for warming fluids includes a heating element disposed on an adhesive strip and operable to be secured to at least an outer portion of a tubing having fluid flowing between an inlet and an outlet, the outlet being proximate a percutaneous point of insertion into a biological interface, and the heating element having a sufficient length to extend from proximate the outlet toward the inlet so as to ensure that the fluid at the outlet is at a desired temperature.

According to another embodiment, an apparatus for warming fluids includes a reservoir to store fluid at a first temperature and operable to release the fluid through an outlet, a tubing having a first end coupled to the outlet and a second end proximate an inlet of a biological interface, the tubing is operable to allow a flow of the fluid between the first end and the second end and a flexible heating element in conductive contact with at least an outer portion of the tubing, the flexible heating element extends from proximate the first end to proximate the second end wherein the fluid reaches the inlet to the biological interface at a second temperature.

According to yet another embodiment, a method for warming fluids includes storing fluid in a reservoir at a first temperature, releasing the fluid through an outlet of the reservoir and into a first end of a tubing coupled to the outlet wherein the fluid flows at least between the first end of the tubing and a second end of the tubing coupled to an inlet of a biological interface, placing a flexible heating element in conductive contact with at least an outer portion of the tubing, the flexible heating element extends from proximate the first end to proximate the second end, and heating the at least outer portion of the tubing that is in conductive contact with the heating element so that the fluid at the inlet to the biological interface is at a second temperature.

### BRIEF DESCRIPTION OF THE SEVERAL VIEWS OF THE DRAWING(S)

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 illustrates a patient being intravenously administered with a heated fluid, the fluid being heated using the heating element according to one embodiment of the invention.

FIG. 2 is a more detailed illustration of the heating element of FIG. 1, according to one embodiment of the invention.

FIGS. 2A-2C are cross-sectional views of the heating element, according to various embodiments of the invention.

FIG. 3A is a top side view of a heating element printed on the adhesive backing material, according to one embodiment of the invention.

FIG. 3B shows the heating element of FIG. 3A applied to a fluid tubing, according to one embodiment of the invention.

FIG. 3C is a partial back side view of the heating element printed on the adhesive backing material of FIG. 3A, according to one illustrated embodiment of the invention.

FIG. 4A is an intravenous fluid delivery system having multiple heating elements coupled thereto, according to one embodiment of the invention.

FIG. 4B is a more detailed view of one of the heating elements illustrated in FIG. 4A, according to one embodiment of the invention.

FIG. 5A is a data table for a heating element of a selected resistance, according to one illustrated embodiment of the invention.

FIG. 5B is a graph illustrating the data of FIG. 5A, according to one embodiment of the invention.

### DETAILED DESCRIPTION OF THE INVENTION

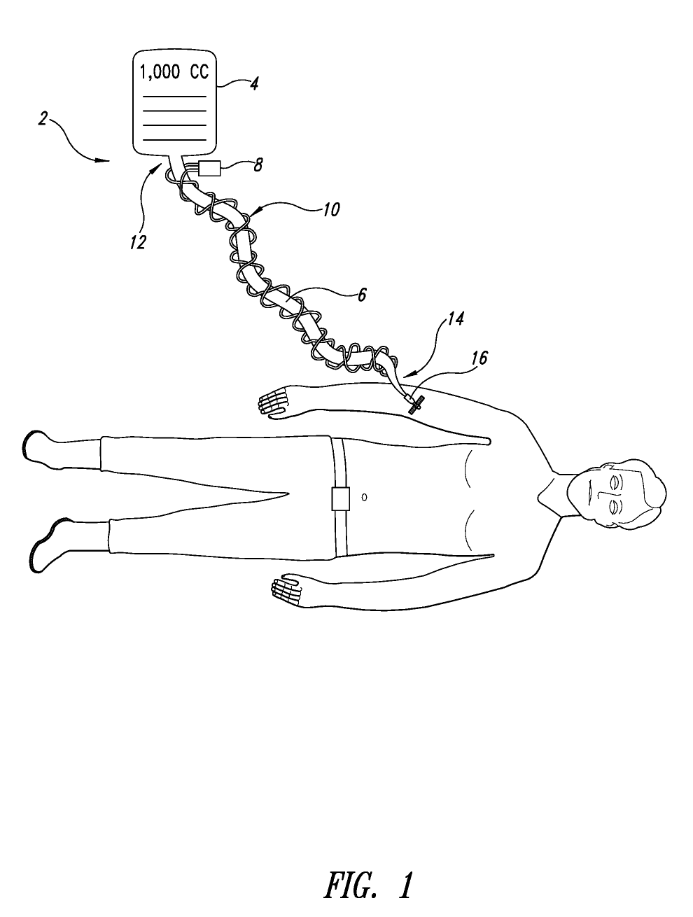

FIG. 1 shows a patient 1 being administered with intravenous (called IV herein) fluid that is heated, according to one embodiment.

The patient 1 may, for example, be a soldier on a battle field receiving heated IV fluid. Alternatively, the patient may be in a remote area without electricity or in an undeveloped country. An IV apparatus 2 to heat fluid therein prior to delivery into the patient 1 comprises an IV bag 4, a fluid line 6, a power device 8 and a heating element 10. The fluid line 6 having a first end 12 coupled proximate the IV bag 4 and a second end 14 positioned opposite the first end 12. The second end 14 may be coupled to a catheter 16 having a needle therein for insertion into a blood vessel of the patient 1. The blood vessel may, for example, be a vein or an artery.

The IV apparatus 2 used to deliver the heated fluid to the patient 1 may be any standard fluid delivery system used in the medical field. The fluid stored in the IV bag 4 flows through the fluid line 6 into the blood vessel of the patient 1. The fluid may be any type of solution used to increase the volume of blood or other fluids in the patient 1. For example, upon loss of blood, it may be desirable to administer blood, plasma water with electrolytes, sugar or a sodium chloride solution into the patient 1.

The heating element 10 is operable to heat the fluid in the fluid line 6 to a safe temperature, prior to entering the blood vessel of the patient 1. The heating element 10 is of a known resistance, usually in the range of 25Ω to 45Ω, in one embodiment 300 and in another 330. It extends along a length of the fluid line 6. The power device 8 is operable to apply a selected voltage across the heating element 10. The power device may, for example, be a low voltage AC or DC power device.

The selected voltage value is set to cause the heating element 10 to heat the fluid to the safe temperature value. The safe temperature value may be approximately in the middle of a safe temperature range of 33° C. to 38° C. The power device 8 may be a battery, capacitor, voltage regulator, or any other power supply.

In one embodiment, the IV fluid heater is comprised of a resistive heating element, a battery, and some adhesive to hold the resistive heating element adjacent the tubing of the fluid. No temperature sensors, electronic controls, voltage regulators, or other structures are required. The resistor, having a known resistance value, will permit a selected amount of current to pass therethrough when a known voltage of the battery is provided. The amount of current which will pass through the resistor is given by Ohm's law, V=IR, where V is voltage, I is current, and R is the resistance of the circuit. Further, the amount of power which is provided for heating the resistor is given in watts according to the equation W=VI, where W is watts, V is voltage, and I is current. The battery or other power source is known to provide a selected, steady state voltage. Since the resistance value is known and does not change, then the amount of current will also be set, and the amount of power provided to the resistor will be a preset, constant value.

The selected voltage applied to the heating element 10 and the resistance of the heating element 10 are held constant, thereby constantly holding a temperature of the heating element 10 at a desired temperature. Since the heating element 10 reaches a known temperature in response to a known amount of applied voltage, the temperature of the fluid in the fluid line 6 can be assured of never exceeding a selected value. In other words, the temperature of the fluid in the fluid line 6 will be determined based on the voltage that is applied to the heating element 10. The voltage is a steady state in one embodiment, so that the temperature will always be at a set value.

The temperature of the human in proper health is approximately 37° C. If fluid is provided to the patient in the temperature range between 33° C. and 38° C., the fluid will nearly match the temperature of the body and will neither raise nor lower the body temperature. If the patient is undergoing shock, the body temperature may tend to drop rapidly. Providing warm fluid at a known temperature will assist to overcome shock and to improve the overall health of the patient.

According to one embodiment of the present invention, the temperature of the resistor is selected to never exceed a value that will raise the level of the fluid to above a safe temperature for administering in an IV. A patient's body has a better tolerance level for receiving fluids which are colder than a body temperature than fluids which are higher than the body temperature. A patient may safely and easily receive fluids 15° C. below the body temperature, for example, fluid in the temperature range of 20° C. to 35° C., and show little to no ill effects. On the other hand, if a patient were to receive blood or other fluid that is in the range of 42° C. to 55° C., severe immediate damage will be done to the patient. At elevated temperatures, even 10 degrees above normal, many blood cells will be destroyed where the heated fluid is entering the patient's body, and permanent damage may be done to cell walls, blood vessels, and body organs. Accordingly, it is acceptable if the fluid is somewhat less than the target temperature of 37° C., but it is unacceptable if the fluid exceeds the target temperature of 37° C. by more than one or two degrees. Accordingly, the temperature which the heating element will reach is selected to be in a range that will heat the fluid to approximately 37.5° C. even if the heater is on at full power for long periods of time, for example several hours. Since the temperature of the resistor cannot exceed a set value, it is assured that the temperature of the fluid will not exceed a safe value.

The IV apparatus 2 advantageously eliminates a need for temperature sensors of the fluid and feedback loops to assure that the fluid temperature does not exceed an unsafe temperature. Since the need for temperature sensors is eliminated, the cost of systems for heating intravenous fluids is reduced. If temperature sensors and feedback loops are needed, the control system that includes multiple components will be costly and require further power.

The inventive apparatus is also quite robust. Since the only components include a simple resistor, an electrical connection of the resistor of a battery, and some technique to hold the heater to the tube, such as an adhesive strip or tape to hold the heater against the fluid tubing, there is nothing to break. Even if the components are subject to impact, crushing, static electricity, or otherwise stressed, the robust resistor will not break and normal batteries, except for severe impact, will still work appropriately and output the selected voltage. Even in a worst-case scenario, the battery voltage may reduce, but it will not increase. The proper temperature is therefore not exceeded, even in a failure mode.

The present invention is drastically different from complex power regulator systems which may include control feedback. Such systems include electronic circuit boards, which, if subjected to impacts, crushing or other stresses, may break one or more components and thus render the system useless. In a worst-case scenario, the temperature sensors and electronic controls may be broken, but the power supply may be effective, and thus overheat the fluid without the physician knowing that the fluid is at a much higher than safe temperature. Thus, the present invention is substantially more robust and is assured of providing the fluid at the proper temperature without failures. Such a system is particularly advantageous in battlefield conditions, field hospitals, undeveloped countries without a well-regulated power supply network, or villages which are distant from any safe or well-known power supply.

The IV apparatus 1 is also advantageous in that the IV apparatus 1 allows somebody not medically trained to administer heated IV fluid to the patient 1 without the risk of overheating the patient 1 and causing irreversible damage.

The heating element 10 may be designed or otherwise selected to allow for a substantially low voltage to cause the heating element 10 to heat the fluid in the fluid line 6 to the safe temperature. The power device 8 may be electrically coupled to the heating element 10 to supply an amount of voltage to the heating element 10 that causes the heating element 10 to heat the fluid to the safe temperature value. In other words, the voltage of the power device 8 is be used to control the fluid temperature in the fluid line 6 and may be relied upon as an indication of the actual fluid temperature in the fluid line 6. The heating element 10 serves itself as a temperature limiting device for heating the fluid to the safe temperature.

Since the temperature of the heater will not exceed a safe temperature, there is some likelihood that as the fluid passes from the bag 4 to the distal end 14, that it may be heated only slightly, but not as high as would be desired. There are a number of solutions to increase the temperature of the fluid as it exits the distal end 14. One technique is to increase the length of the tubing in the fluid line 6. The heating element 10 extends along the entire fluid line 6, and if a longer fluid line is provided, then this will give more time for the fluid to reach a higher temperature. Another solution is to flow the temperature more slowly through the tubing 6. If the fluid is traveling very slowly or at a standstill, then it can be assured of heating to the correct temperature as it passes into the patient from the bag 4 through the tubing 6. Even if the fluid is traveling very slowly, as may be the case in some instances, the physician can be assured that the fluid will not overheat but will be at exactly the correct temperature as it enters the body.

There may be some instances in which the fluid line is a set length which cannot easily be increased, and the fluid must be provided at a high rate of speed to the patient, such as a blood transfusion which is rapidly needed. In such situations, another technique which may be used is to attach multiple heating elements 10 to the fluid line 6. Each of the heating elements will reach the desired temperature and heat the fluid line. If a larger number of heating elements are used, a greater amount of heat will be introduced into the fluid and it will reach the proper temperature more quickly. The various heating elements may be provided with their own power supply, respectively, or alternatively may be attached to the same power supply 8 which will provide the set voltage to all the heating elements. Thus some embodiments of the invention may advantageously include multiple heating elements coupled along the length of fluid line 6 and/or the IV bag 4 such that the desired fluid temperature is reached in a short amount of time. Due to the temperature-limiting nature of the IV apparatus 2, each heating element 10 cannot cause the fluid temperature to be above the safe temperature. The multiple heating element design may be implemented using a common power source or alternatively multiple power sources may be electrically coupled to respective heating elements. While a battery that outputs a preset voltage is an acceptable power supply, any power regulator that is capable of being set to output a selected voltage can be used.

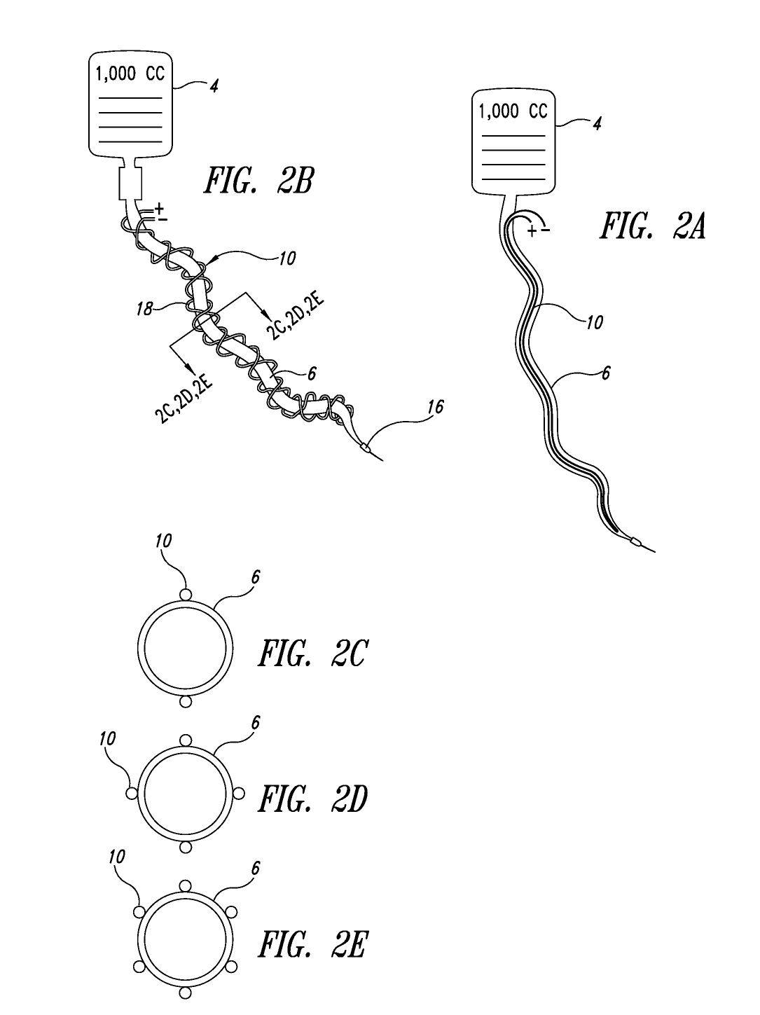

FIGS. 2A and 2B show schematic illustrations of the heating element 10, according to various embodiments.

In FIG. 2A, the heating element 10 comprises a flexible material in the form of a thread that is capable of extending along the length of the fluid line 6. The flexible material may take the form of stainless steel fiber strands or other fibers or threads having resistant characteristics such as, for example, a polyester carbon blend. As illustrated in FIG. 2A, the heating element 10 may be placed in a straight line along an external portion of the fluid line 6 such that portions of the heating element 10 do not overlap. A piece of tape or other adhesive attached the heater thread 10 to the tube 6. In this embodiment of FIG. 2A, the heating element 10 may be a single simple thread such as a stainless steel fiber or polycarbonate thread.

In military conditions, the resistor can easily be provided at a very low cost to every soldier. It may be a small piece of thread provided in a first aid kit. Alternatively, it may be a piece of thread provided in all batteries, cell phones or radios, since such electrical components also have a battery. The resistance of the thread will be selected based on the power supply with which it is to be used. For example, thread to be used with a cell phone battery, which is traditionally 3.7 volts, would have a resistance selected to provide the proper temperature for that voltage.

The threads are appropriately marked with the correct voltage which is to be applied to them. For example, the threads may be marked with 3.7 volts, which is a common cell phone battery voltage, or alternatively with lower voltage levels, such as 1.5 volts or 3 volts of the type commonly available in flashlight batteries, or higher voltage such as 6 volts or 12 volts, at a level normally provided by high-powered radios, automobiles, and DC power supplies. Thus, at a very low cost, every soldier is able to carry with him a heating element.

At the lightest-weight, most simple form, the resistor is a bare conductor, such as a stainless steel fiber, polycarbonate, polyester carbon blend, or other moderately conductive thread. In such applications it will be important for the user to ensure that the thread does not overlap or cross when being placed along the tube. This would create a shorter path from the positive terminal to the negative terminal of the battery, and thus increase somewhat the current. Of course, since the resistor would have a shorter length, the amount of heat which could be generated for heating the fluid would be correspondingly less, even though the wire may temporarily reach a somewhat higher temperature than if the entire length of the wire is used. Overlapping portions of the heating element 10 may cause a variation in resistance that changes the amount of heat dissipated into the fluid line 6, thereby varying the temperature of the fluid in the fluid line 6. Thus, aligning the heating element 10 along the fluid line 6 such that no overlap occurs assures that the fluid remains at the safe temperature while the heating element 10 is applied with the selected voltage.

Alternatively and/or additionally, as illustrated in FIG. 2B, the heating element 10 may be encased within an insulative sleeve 18 such that an overlap of the heating element 10 will not cause a change in fluid temperature.

According to one embodiment, the heating element 10 coated with an insulator or encased within the insulative sleeve 18 may be braided along the length of the fluid line 6. The heating element 10 may be braided such that overlap occurs at multiple points along the length of the fluid line 6. For example, the heating element 10 may be shrink-wrapped to the fluid line 6. Such may be implemented by sliding the fluid line 6 into a shrink-wrap tubing and applying heat to seal the shrink-wrap tubing around the fluid line 6 and the heating element 10. Alternatively, the fluid line 6 may be sealed with other types of film that may be used to secure and protect the heating element 10 proximate the fluid line 6. The shrink-wrapped fluid line 6 may be sterilized prior to use.

As mentioned above, multiple heating elements may be coupled to or otherwise adhered to the fluid line 6. Such is illustrated in the cross-sectional views of FIGS. 2C-2E. As mentioned above, since each heating element 10 limits the fluid temperature to the safe temperature, there is substantially no risk of overheating the fluid.

As shown in FIG. 2C, the heating element 10 may include two ends (e.g., a positive and a negative end) next to each other and respectively coupled to power outputs of the power device 8. As illustrated in FIGS. 2A and 2B, the heating element 10 may extend from proximate the IV bag 4 to proximate the insertion point of the catheter 16. As such, the fluid leaving the IV bag 4 is substantially unaffected by outside temperature, thereby assuring that the fluid reaches the patient 1 at a desired safe temperature. Implementing the heating element 10 using the stainless steel fiber allows for the power device 8 to be of a low voltage power device to power the heating element 10 such that the desired fluid temperature is reached.

Some embodiments of the IV apparatus 2 may have some additional components to limit the voltage applied to the heating element 10 such that the fluid temperature proximate the second end 14 does not exceed the safe temperature. For example, according to one embodiment, one place in the electric circuit, such as the top of the heater 10 or the power device 8, may include a fuse to limit the amount of voltage applied to the heating element 10 to an amount above which the fluid temperature would not exceed the safe temperature. In the embodiment of FIG. 2D, two separate heating elements are provided along the length of the tube which can more rapidly heat the fluid to the proper temperature. Thus, the fluid can either flow more quickly through the tube or the fluid may start at a lower temperature and reach the desired temperature more quickly since two heating elements are provided. As illustrated in FIG. 2E, three or more heating elements may also be provided to provide even more heat to the fluid. Alternatively and/or additionally, a converter (not shown) may be coupled to an output of the power device 8 to convert the output voltage to a voltage which causes the heating element 10 to heat the fluid in the fluid line 6 at the safe temperature. Thus, the IV apparatus 2 may be advantageously designed such that regardless of the applied voltage, the fluid in the fluid line 6 is at the safe temperature.

INSERT H: For example, the heater thread or wire is designated as a 6-volt wire, it may have a fuse or circuit breaker therein, which would create an open circuit if a voltage higher than 7 volts is placed on the line. Therefore, if a user accidentally attaches a heating element designed for 6 volts to a 12-volt supply, the resistor will automatically become open circuit and not heat at all. Of course, it is acceptable to provide a voltage lower than the designated voltage to the line, and some heating will occur, although not at the optimum level.

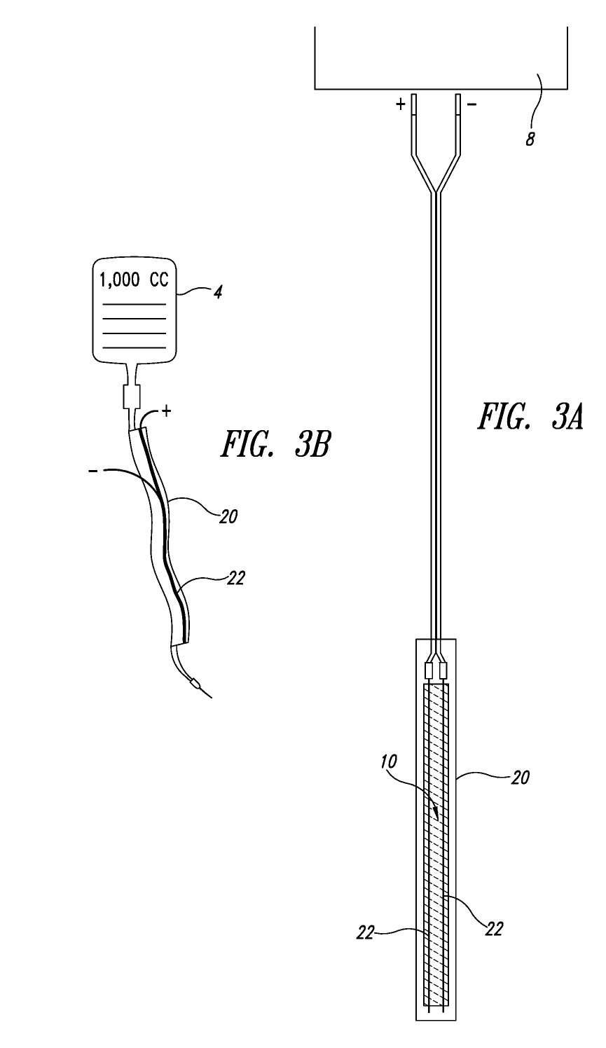

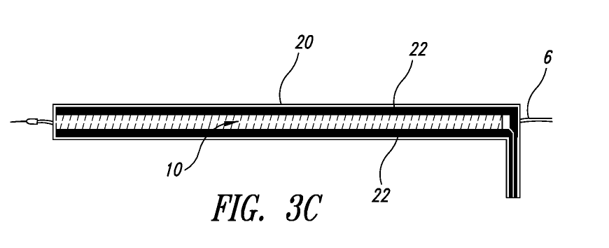

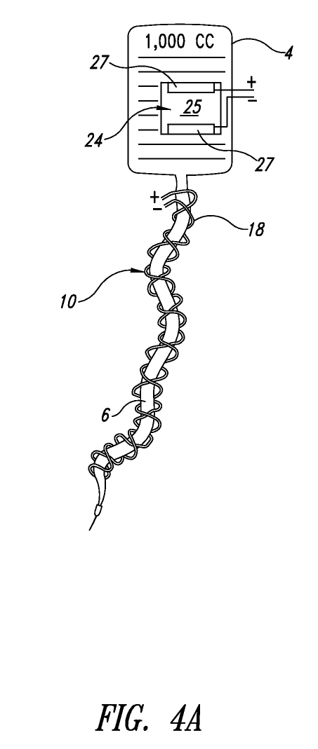

FIG. 3A shows the heating element 10 printed on an adhesive backing material 20 adhered to the fluid line 6, according to one illustrated embodiment. As shown in FIG. 3A, highly conductive bus bars 22 are provided coupled to positive and negative power supplies, respectively. These are positioned by an adhesive layer 20. A conductive layer is then placed across the bars 22 so as to generate heat as the current passes from one conductive bar 22 to the other conductive bar 22. In this embodiment, the heat is generated between two closely adjacent conductors and the heating resistive element 10 is equally heated along its entire length. An adhesive backing material 20 holds the entire resistive heater. It has a high integrity adhesive on the side to which the resistor is attached. This permits the resistor to be folded so that the two edges of the adhesive touch each other so as to make a loop that can be completely wrapped around the fluid line, as shown in FIG. 3B.

FIG. 3B shows the adhesive backing material 20 having the heating element 10 thereon being electrically coupled to the power device 8, according to one illustrated embodiment. The backing 20 is wrapped around the fluid line 6, encasing it like a blanket or sleeve. FIG. 3C shows a detailed view of the adhesive backing material 20 having the heating element 10 printed thereon, according to one illustrated embodiment.

The adhesive backing material 20 may be layered with conductive material 22 that takes the form of bus bars to conduct electricity. For example, the conductive material 22 may be designed as two parallel lines with a gap therebetween. The gap may be screen printed with the heating element 10 so as to electrically couple the bus bar edges and function as a resistive material. The heating element 10 may, for example, have a silver filled ink. It may also be graphite strands, carbon laminate, carbon fiber laminate, polyester or other resistive metal and materials. Outputs of the power device 8 may be electrically coupled to respective bus bars (e.g., the conductive material 22) on the adhesive backing material 20 to provide voltage to heat the fluid to the safe temperature.

During use, the adhesive backing material 20 may be coupled or otherwise adhered to the fluid line 6 as shown in FIGS. 3B and 3C. The adhesive backing material 20, the conductive material 22 thereon and the printed heating element 10 may be of a flexible material such that it may bend or otherwise reshape according to a size, shape or form of the fluid line 6. Such flexible design prevents the conductive material 22 from becoming uncoupled and thereby altering the resistance of the heating element 10. The adhesive backing material 20 may be folded about a center length of the heating element and enclose the fluid line 6 therein. The adhesive backing material 20 having the heating element 10 printed thereon may extend along the length of the fluid line 6, from proximate the first end 12 to the distal second end 14.

Outputs of the power device 8 may be electrically coupled to the respective conductive material 22 (e.g., bus bars) on the adhesive backing material 20. The heating element 10 is electrically coupled to the fluid line upon being adhered to the adhesive backing material 20 along the length of the fluid line such that the printed heating element 10 of the adhesive backing material 20 encases the fluid supply line along the length thereof.

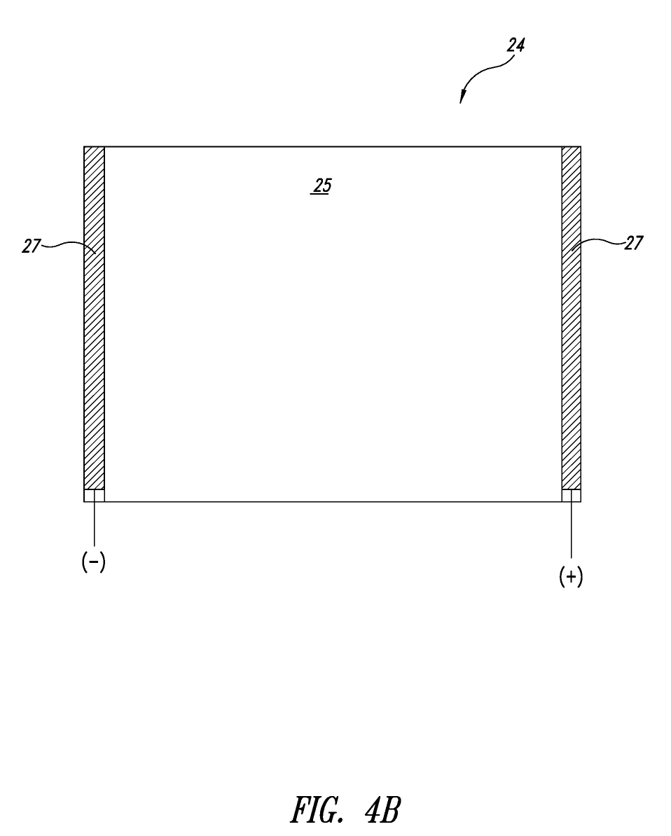

FIG. 4A shows multiple heating elements used to heat the IV fluid, according to one illustrated embodiment.

In addition to having the heating element 10 electrically coupled along the length of the fluid line 6, some embodiments may include at least one additional heating element 24 coupled to the bag 11 of the IV. The additional heating element 24 may be adhered to at least one of the fluid line 6 and the IV bag 4. The additional heating element 24 may heat to the same temperature as the heating element 10 coupled to the fluid line 6, although the dimensions of the additional heating element 24 may be of different size, shape or form than the heating element 10.

As illustrated in FIG. 4B, the additional heating element 24 may, for example, be of a postcard size (e.g., 13 cm×20 cm). The additional heating element 24 may be printed on an adhesive backing material 25 operable to be adhered to the IV bag 4. The adhesive backing material 25 may be layered with conductive material 27 that takes the form of bus bars to conduct electricity. For example, the conductive material 25 may be designed as two parallel lines with a gap therebetween. The gap may be screen printed with the additional heating element 24 so as to electrically couple the bus bar edges and function as a resistive material. The additional heating element 24 may, for example, take the form of a silver based conductive ink, graphite, carbon fibers, or other resistive materials.

The power device 8 may be electrically coupled to respective bus bars on the adhesive backing material 25 to provide voltage to heat the fluid in the IV bag 4 to the safe temperature. Alternatively, an additional power device may be electrically coupled to the additional heating element 24 to heat the fluid to the safe temperature.

The resistance of the heating element 10 and of the additional heating element 24 may be selected so that having substantially same voltage supplied to each, to cause each of the heating elements 10, 24 to heat the fluid to the safe temperature. Thus, having the additional heating element 24 allows for the fluid to reach the safe temperature at a faster rate since the bag itself is also heated.

In other embodiments, the flow rate of the fluid may be varied to control an amount of time before the fluid reaches the safe temperature. For example, the flow rate may be reduced to allow for the heating element 10 to heat the fluid to the safe temperature in a shorter amount of time. Alternatively and/or additionally, multiple heating elements 10 may be adhered to the fluid line 6 to further reduce the amount of time before the fluid reaches the safe temperature. It will be appreciated by those skilled in the art that although the IV apparatus 2 may include multiple heating elements 10, 24 the temperature of the fluid will not exceed the safe temperature since each heating element 10, 24 may have substantially same resistance and be applied with same voltage to cause the heating elements 10, 24 to heat to the safe temperature.

The resistance of each of the heating element 10 and the additional heating element 24 may, for example, be approximately 33Ω. The power device 8 coupled to at least one of the heating elements 10, 24 may apply a voltage to cause the heating elements 10, 24 to reach a safe temperature of 39° C. Since the heater is coupled to the tube, the fluid in the tube will reach a maximum temperature of 37.5° C. Other embodiments may heat the IV fluid to a different temperature in the range of 35° C. to 38° C. It may be advantageous to limit heating the IV fluid to the temperature of 39° C. as a precaution for a no-flow event. In other words, if the heating elements 10, 24 are activated and IV fluid is not being administered to the patient 1 while the IV bag 4 is, for example, in the sun or under a blanket, the temperature of the fluid prior to delivery may increase. When fluid is administered into the patient 1 proximately after the no-flow event, the fluid will still be at a safe temperature.

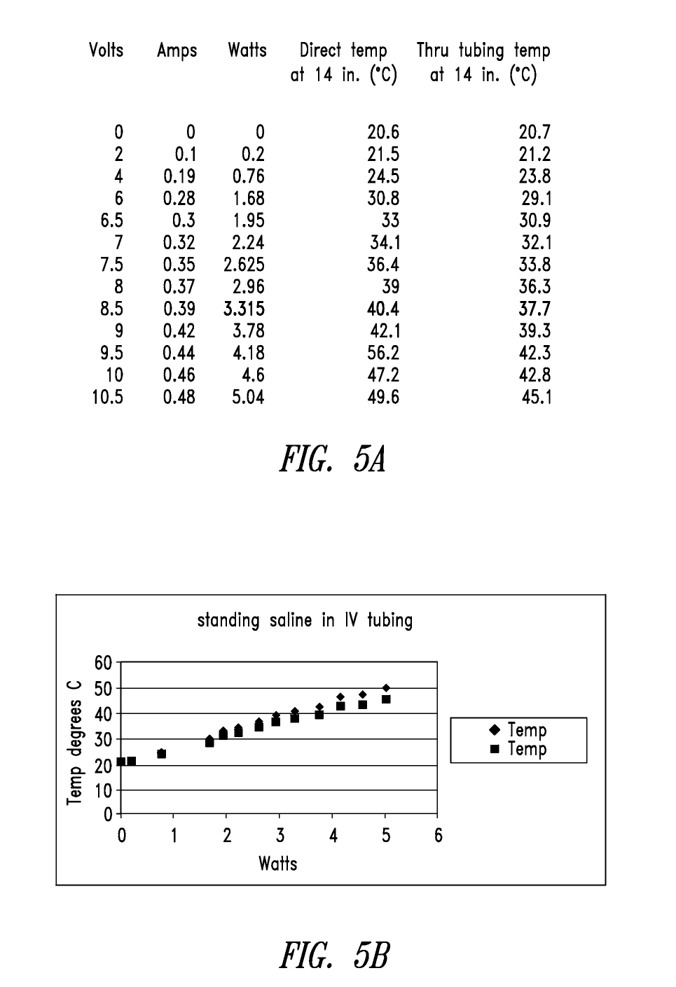

FIGS. 5A and 5B show experimental data of a heating element, according to one embodiment.

In the example of FIGS. 5A and 5B, the actual characteristics of one acceptable stainless steel fiber has been provided. In this example, the stainless steel fibers were provided from Bekerat Corp. of the Netherlands have been found to perform to requirements. Stainless Steel fiber strands are utilized in various designs and given low wattage and low voltage DC to achieve warming.

As shown in FIG. 5A, a length of 35 centimeters (14 inches) of the stainless steel fiber is provided. It is assumed that the ambient temperature is approximately 21° C. In this example, with no power applied, the temperature of the stainless steel fiber is approximately room temperature, as is the temperature of the fluid in the tubing. When a first voltage is applied, for example 2 volts, a small amount of current runs through the fiber and 0.2 watts is provided. This heats the fiber to 21.5° C. If a higher voltage, for example 6 volts, is provided, then 0.28 amps flows through the stainless steel fiber which heats the resistor to 30.8° C. and the fluid in the tubing to approximately 29.1° C. If 8 volts is provided to this particular length of stainless steel thread, then the temperature of the thread reaches 39° C., the fluid in the tube reaches approximately 36.3° C., and approximately 2.96 watts is expended. For this particular length of stainless steel fiber, if 9 volts is supplied, then, as can be seen from the table of FIG. 5A, the fluid reaches approximately 39.3° C. Accordingly, for this particular length of stainless steel fiber, applying a voltage anywhere between the range of 6 volts to 9 volts will result in heating of the fluid to a higher level to provide better therapy for the patient and will keep the temperature below a safe level. As previously stated, the length of the stainless steel fiber can be a different length besides 35 centimeters, which would change the amount of power consumed and appropriately vary the temperature which the wire heats to. FIG. 5B illustrates an example of measuring the temperature of the heater as compared to the temperature of the fluid for standing saline fluid in the IV tubing. In this example, there is no flow of the fluid, and the temperature of the resistor as well as the temperature of the fluid is graphed based on the watts applied. The temperature of the fluid is shown in a block and the temperature of the resistor as a diamond. As can be seen, at approximately 3 watts of power, the temperature of the heater is in the range of 40° C., and the temperature of the saline fluid in the IV tubing is approximately 37° C. Corresponding plots and graphs can easily be created for different resistive materials. Other fibers of resistant characteristics can be utilized to achieve alike form and function i.e., a polyester carbon blend.

In one embodiment, the power connection is a ribbon connector joined to the standard 2 pin CUI wiring and connector as universally accepted by the power unit. This connection of ribbon to wire is a product sold under the trade name CRIMPFLEX®.

It is also designed to provide a constant voltage without the need for feedback and a control or sensor input, as described in more detail herein.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not all referring to the same embodiment. Furthermore, the particular features, structures, or characteristics may be combinable in any suitable manner in one or more embodiments.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

I claim:

1. A method of warming an intravenous fluid comprising:
attaching an electrical resistive heating element to a fluid supply line, a resistance value of the heating element being selected to limit a current through the heating element to a known value for a selected voltage; and
electrically coupling a power source to the electrical resistive heating element, the voltage of the power source having a substantially steady state value that is selected based on the resistance of the heating element, to ensure that the temperature of the heating element cannot exceed a temperature range that is above a safe value for heating the fluid to be intravenously delivered to a patient.

2. The method of claim 1 wherein attaching the electrical resistive heating element includes winding the heating element along a length of the fluid supply line.

3. The method of claim 2 wherein attaching the electrical resistive heating element includes winding a stainless steel thread along the length of the fluid supply line.

4. The method of claim 1 wherein attaching the electrical resistive heating element includes braiding the heating element along a length of the fluid supply line, the heating element encased within an insulative sleeve.

5. The method of claim 4 wherein attaching the electrical resistive heating element includes braiding a stainless steel thread along the length of the fluid supply line, the stainless steel thread encased within the insulative sleeve.

6. The method of claim 5 wherein attaching the electrical resistive heating element includes encasing the fluid supply line having the stainless steel braids thereon with a shrink wrap tubing and applying heat to seal the shrink wrap tubing around the fluid supply line and the stainless steel braids.

7. The method of claim 1, further comprising printing the heating element on an adhesive backing material layered with conductive material that serve as buss bars.

8. The method of claim 7 wherein printing the heating element on the adhesive backing material includes printing at least one of a silver ink, graphite and carbon fibers on the adhesive backing material.

9. The method of claim 7 wherein attaching the electrical resistive heating element to the fluid supply line includes adhering the adhesive backing material along a length of the fluid supply line such that the printed heating element portion of the adhesive backing material encases the fluid supply line along the length thereof.

10. The method of claim 1, further comprising the resistance value of the heating element is approximately 30Ω.

11. The method of claim 1, further comprising limiting the current through the heating element to less than 5 amps.

12. The method of claim 10, further comprising selecting the voltage of the power source not to exceed 6V such that the temperature of the heating element does not exceed 39° C.

13. The method of claim 1, further comprising:
attaching at least one additional resistive heating element to the fluid supply line having a same resistance as the electrical resistive heating element; and
coupling an additional power source to the additional heating element, a voltage of the additional power source having a value that does not exceed a threshold voltage to ensure that the temperature of the heating element cannot exceed a temperature range that is above a selected value relative to the target temperature of a human patient for heating the fluid to be intravenously delivered to the patient.

14. The method of claim 13, further comprising selecting the voltage of the additional power source not to exceed 6V such that the temperature of the heating element does not exceed 39° C.

15. A method for warming fluids, the method comprising:
providing a heating element having a resistance value that limits an amount of heat dissipated from the heating element to a threshold temperature value in response to a specific electric voltage being applied to the heating element;
coupling the heating element to a fluid carrying member having fluid to be delivered into a biological interface to at least partially cause a transfer of heat from the heating element to the fluid; and
controlling a temperature of the fluid so as not to exceed the threshold temperature value by applying the specific voltage to the heating element; and
preventing the voltage of the electrical energy applied to the heating element from being exceeded.

16. The method according to claim 15 wherein the voltage is prevented from exceeding the specific value by a voltage regulator circuit.

17. The method according to claim 15 wherein a circuit breaker is provided and that will create an open circuit if the voltage is exceeded.

18. A method for intravenously feeding fluid to a patient comprising:
placing a needle into a blood vessel of a patient;
providing a flow of intravenous fluid to the needle for supply into the patients blood vessel;
heating the fluid as it flows towards the patients blood vessel; and
preventing the temperature of the heat source that provides heat to the fluid from exceeding a selected value for fluid to be supplied to a patient, by limiting the voltage that can be applied to a resistive heating element to a obtain a temperature of the fluid at or less than 40° C. at all times.

19. An apparatus for warming fluids, the apparatus comprising:
a heating element having a resistance value that limits an amount of heat dissipated from the heating element to a threshold temperature value in response to a specific voltage being applied to the heating element;
a fluid carrying member having the heating element coupled thereto to transfer heat from the heating element to a fluid within the fluid carrying member; and
a power supply to control a temperature of the fluid the power supply limiting the maximum temperature that the fluid can achieve by limiting the voltage applied to the heating element.
